(19) United States
(12) Patent Application Publication
(10) Pub. No.: US 2015/0302160 A1
(43) Pub. Date: Oct. 22, 2015

(54) Method and Apparatus for Monitoring Diet and Activity

(71) Applicant: The Board of Regents of the NV Sys of Higher Edu, Las Vegas NV on Behalf of the Univ of NV Las Vega, Las Vegas, NV (US)

(72) Inventors: Venkatesan Muthukumar, Henderson, NV (US); Jillian Inouye, Henderson, NV (US); Mohamed B. Trabia, Las Vegas, NV (US)

(21) Appl. No.: 14/692,681

(22) Filed: Apr. 21, 2015

Related U.S. Application Data
(60) Provisional application No. 61/982,165, filed on Apr. 21, 2014.

(51) Int. Cl. G06F 19/00; G01N 21/25; A61B 5/16; A61B 5/00; A61B 5/0205; A61B 5/11

(52) U.S. Cl. CPC G06F 19/3406; G06F 19/3475; A61B 5/02055; A61B 5/1118; A61B 5/165; A61B 5/4806; A61B 5/742; G01N 21/255; A61B 5/024

(57) ABSTRACT

A method and apparatus includes a memory, a display and user input module, a camera and micro spectroscopy module, and one or more communication modules communicably coupled to a processor. The processor is configured to capture one or more images and spectroscopy data of the food(s) using the camera and micro spectroscopy module, determine a food type and food amount for each of the food(s) using the image(s) and spectroscopy data, perform a dietary analysis of the food(s) based on the food type and food amount determined from the image(s) and spectroscopy data, determine the set of nutritional data for the food(s) based on the dietary analysis, and provide the set of nutritional data for the food(s) to the memory, the display and user input module or the one or more communication modules.

### CROSS-REFERENCE TO RELATED APPLICATIONS

This application claims priority to and is a non-provisional patent application of U.S. Provisional Application No. 61/982,165, filed Apr. 21, 2014, the contents of which is incorporated by reference in its entirety.

### INCORPORATION-BY-REFERENCE OF MATERIALS FILED ON COMPACT DISC

None.

### TECHNICAL FIELD OF THE INVENTION

The present invention relates in general to the field of electronics and nutrition, and more specifically to a method and apparatus for monitoring diet and activity.

### STATEMENT OF FEDERALLY FUNDED RESEARCH

None.

### BACKGROUND OF THE INVENTION

Without limiting the scope of the invention, its background is described in connection with nutrition. One of the main purposes of dietary assessment is to evaluate usual food and nutrient intakes of population groups relative to dietary recommendations. The usual intake reflects the long-run average of daily intake, yet reliable estimations based on short-term dietary assessment methods continue to be an important challenge. The U.S. Department of Agriculture 5-pass method, 24-hour dietary recall is considered to be the gold standard for all dietary assessment associated with research activity. [1,2] This is a 5-step dietary interview that includes multiple passes through the 24 hours of the previous day, during which respondents receive cues to help them remember and describe foods they consumed. In large samples food frequency questionnaires (FFQs) are usually used to assess dietary intake over a specified time period due to low cost and ease of administration. [3] Furthermore due to its self-report nature, it is recognized that under-reporting of dietary intake is fairly common, particularly in individuals who are overweight, [4-7] have diabetes, [7] and are wanting to reduce their weight. [4] FFQs have been used to show significant differences in energy intake across a 12-month period in randomized conditions that received different dietary prescriptions. [8] Other research suggests that while self-reported energy intake from FFQs may contain errors, macronutrient reporting, particularly that adjusted for energy intake (i.e., percent energy from macronutrients or gram intake of macronutrients per 1000 kcal), may be less prone to reporting-error. [9] This suggests that the findings of changes in relative intake of macronutrients may be more accurate than changes in absolute energy intake. All self-report methods are challenging because people do not commonly attend to the foods they have eaten; nor remember everything consumed. They often do not know the contents of the foods eaten and cannot estimate portion sizes accurately or consistently. Additionally accuracy appears to be associated with gender and body size. [10] A long history of technological innovation in the design and development of diet assessment systems has evolved over time to address the accuracy of dietary intake assessment that can lead to improved analytical and statistical interpretations. Computers and related technology have facilitated these initiatives. Food intake computer software systems, cell phones with camera capability and voice recognition linked to food-nutrient databases, and wearable data recording devices have been designed and implemented in an attempt to meet some of the challenges of dietary assessment. Successful development of these projects will further enhance the accuracy, reduce cost, and minimize respondent burden of existing diet assessment systems. [11]

Current non-intrusive solutions for monitoring exercise activity and dietary intake include systems like Sensecam, SCiO—molecule sensors, Healbe GoBe—calorie intake measurement watch, Tellspec—chemical composition analyzer, and mobile phone apps that use the built-in camera. The Sensecam system does not record user exercise activity or vital signals. It continuously records user action and requires the user to select the image or image sequence that contains food consumed. This post-process can be time consuming and irregular based on the user. Also, privacy issues are involved due to the continuous recording of the surroundings. Fitbits, Jawbone UPs, and Nike FuelBands accounted for 97 percent of all smartphone-enabled activity trackers sold in 2013. Fitness and wellness devices such as Fitbit, only monitor exercise activity and some devices monitor heart rates. Calorie intake and expenditures are done using their software tool, which requires inordinate time for data input and is inaccurate because of user memory lapses between the times of consumption and data input. The SCiO and Tellspec systems use near infrared wavelength sensing to determine the composition of the food, but cannot determine food quantity and multi-food calorie intake. The Healbe GoBe system uses infrared techniques to measure calorie intake through the skin.

### SUMMARY OF THE INVENTION

This present invention merges the missing link between the different lines of products discussed above. The present invention combines the functionality of monitoring daily exercise activity and nutritional or dietary intake using a single device. Automatic classification of food consumed and determining calorie intake is a daunting task and can be only done using expert systems. Therefore, the present invention captures the quantity of food consumed, determines the different types of food and portion of food and estimates the quantity. Food quality estimates can also be determined. The nutritional data, such as calories, will be estimated based on the automatic classification of food based on images, near infrared spectroscopy sensors, and/or audio and text inputs to augment the type of food and fat content intake. As a result, the present invention provides a device and method that enables diabetic and in general other weight watchers to monitor their exercise activities, sleep patterns, and food/calorie intake more efficiently and non-intrusively. The device can be interfaced with application software for extracting and visualizing collected data.

More specifically, the present invention provides an apparatus that includes a portable device housing, a microcontroller or processor disposed within the portable device housing, a memory disposed within the portable device housing and communicably coupled to the processor, a display and user input module disposed on the portable device housing and communicably coupled to the processor, a camera disposed on the portable device housing and communicably coupled to the processor, a near infrared spectroscopy sensor or module disposed on the portable device housing and communicably coupled to the processor, and one or more communication modules disposed on or within the portable device housing and communicably coupled to the processor. The processor is configured to capture one or more images of the one or more foods using the camera and near infrared spectroscopy data of the one or more foods using the near infrared spectroscopy module, determine a food type and a food amount for each of the one or more foods using the one or more images and near infrared spectroscopy data, perform a dietary analysis of the one or more foods based on the food type and food amount determined from the one or more images and near infrared spectroscopy data, determine the set of nutritional data for the one or more foods based on the dietary analysis, and provide the set of nutritional data for the one or more foods to the memory, the display and user input module or the one or more communication module.

In addition, the present invention provides a computerized method for providing a set of nutritional data for one or more foods that includes the steps of providing a portable device, capturing one or more images and spectroscopy data of the one or more foods using a camera and micro spectroscopy module, determining a food type and a food amount for each of the one or more foods using the one or more images and the spectroscopy data, performing a dietary analysis of the one or more foods based on the food type and food amount determined from the one or more images and spectroscopy data, determining the set of nutritional data for the one or more foods based on the dietary analysis, and providing the set of nutritional data for the one or more foods to a memory, a display and user input module or one or more communication modules. The portable device includes the memory, the display and user input module, the camera and micro spectroscopy module and one or more communication modules communicably coupled to the processor.

### BRIEF DESCRIPTION OF THE DRAWINGS

For a more complete understanding of the features and advantages of the present invention, reference is now made to the detailed description of the invention along with the accompanying figures and in which:

FIG. 1 is a block diagram of a portable device for providing a set of nutritional data for one or more foods in accordance with one embodiment of the present invention;

FIG. 2 is a rendition of a portable device in accordance with one embodiment of the present invention;

FIG. 3 is a flow chart of a method for providing a set of nutritional data for one or more foods in accordance with one embodiment of the present invention;

FIGS. 4A-4F are photographs illustrating taking one or more images of food in accordance with one embodiment of the present invention;

FIG. 5 is a flow chart of an image capture process in accordance with one embodiment of the present invention; and

FIG. 6 is a flow chart of an image analysis process in accordance with one embodiment of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The device, which is similar to a wristwatch, monitors and tracks exercise activity, sleep patterns, and heart-rate using embedded electronics and is equipped with a camera and near infrared (NIR) spectroscopy sensor or module to capture dietary intake. The device can be equipped with low power Bluetooth module (BLE) for communication and a micro USB interface for battery charging and configuration purpose. An application captures nutritional information of food intake and a dietary analysis of the food captured in images from the camera and NIR spectroscopy module along with the information on exercise and sleep.

Now referring to FIG. 1, a block diagram of an apparatus 100 for providing a set of nutritional data for one or more foods in accordance with one embodiment of the present invention is shown. The apparatus 100 includes a portable device housing, a microcontroller or processor 102 disposed within the portable device housing, a memory 104 (e.g., a high density, low latency memory storage) disposed within the portable device housing and communicably coupled to the processor 102, a display and user input module 106 disposed on the portable device housing and communicably coupled to the processor 102, a camera and micro spectroscopy sensor or module 108 disposed on the portable device housing and communicably coupled to the processor 102, one or more communication modules 110 disposed on or within the portable device housing and communicably coupled to the processor 102, and a power and battery management module 112 (e.g., battery, etc.). The processor 102 is configured to capture one or more images and spectroscopy data of the one or more foods using the camera and micro spectroscopy sensor or module 108, determine a food type and a food amount for each of the one or more foods using the one or more images and spectroscopy data, perform a dietary analysis of the one or more foods based on the food type and food amount determined from the one or more images and spectroscopy data, determine the set of nutritional data for the one or more foods based on the dietary analysis, and provide the set of nutritional data for the one or more foods to the memory, the display and user input module or the one or more communication modules. Food quality estimates can also be determined. Audio and text inputs can also be used to augment the type of food and fat content intake. Note that the camera and micro spectroscopy sensor 108 can be separate components.

Components and modules described herein can be communicably coupled to one another via direct, indirect, physical or wireless connections (e.g., connectors, conductors, wires, buses, interfaces, buffers, transceivers, etc.). For example, the microcontroller or processor 102 can be connected and communicate with the following components and modules through a high speed bus 118: memory 104; display and user input module 106; camera module 108; sensor(s) 114; microphone and speaker module 116; and communication module 110.

The apparatus 100 may also include one or more sensors 114 (e.g., accelerometer, a heart rate monitor, a thermometer, etc.) disposed on or within the portable device housing and communicably coupled to the processor 102 such that the processor 102 is configured to monitor one or more biological indicators of a user (e.g., an exercise activity, a sleep pattern, a stress level, a temperature, etc.) using the one or more sensors 114. The processor 102 can also be configured to analyze the one or more biological indicators and provide a result of the analysis of one or more biological indicators to the memory, the display and user input module or the one or more communication modules. In addition, the apparatus 100 may include a microphone, speaker or tone generator 116 communicably coupled to the processor 102, a global positioning module disposed within the portable device housing and communicably coupled to the processor 102, and/or a power supply recharger (e.g., recharging port connected to a battery, a battery recharger connected to a battery that recharges the battery using electromagnetic fields, motion or solar energy, etc.).

The processor 102 can also be configured to store the one or more images and spectroscopy data, store the set of nutritional data, confirm the food type and/or the food amount, request an additional information from a user, or any other desired functionality. Note that the processor 102 can be configured to transmit the one or more images and spectroscopy data to a remote device (e.g., a portable computing device, a desktop or laptop computer, a mobile phone, an electronic tablet, a server computer, etc.) for processing and analysis, and receive the set of nutritional data from the remote device. The processor 102 may also have a clock function, a timer function or both.

In addition, the apparatus may include a flash, a laser, an infrared proximity sensor disposed on the portable device housing and communicably coupled to the processor 102 or the camera 102. The display and user input module 106 may include one or more displays, one or more buttons, one or more touch screen, or a combination thereof. The one or more communication modules 110 may include a wireless communication module, an infrared communication module, a cable connector, or a combination thereof.

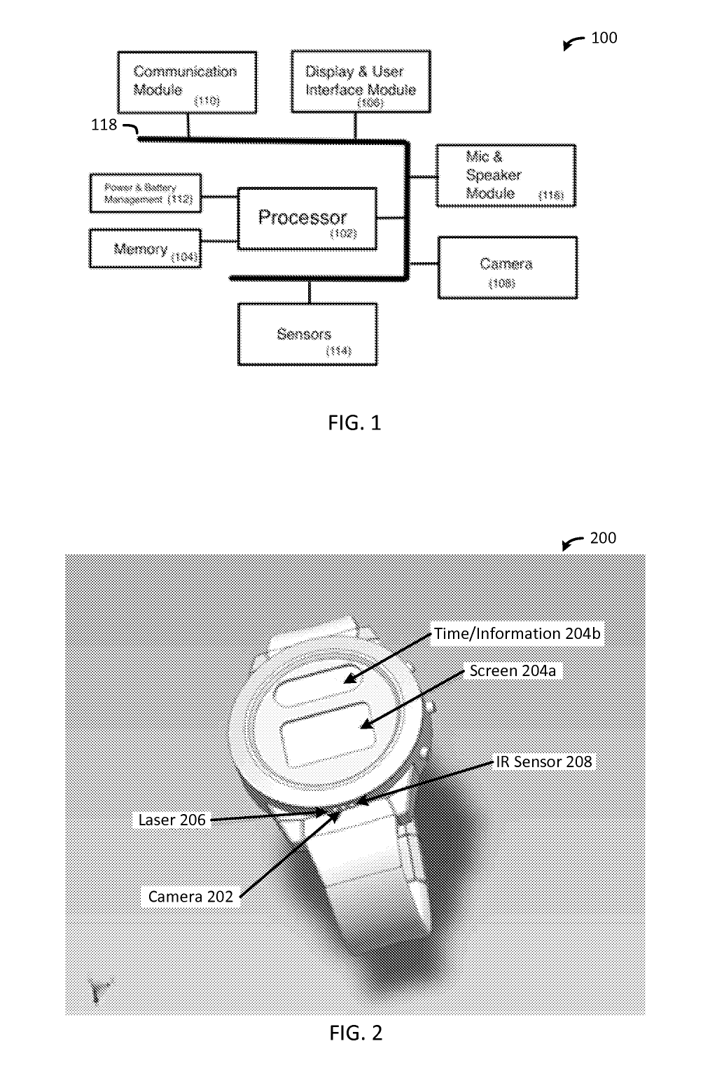

Referring now to FIG. 2, a photograph of a non-limiting example of a portable device 200 in accordance with one embodiment of the present invention is shown. The portable device 200 is a wearable device (wristwatch) that monitors and tracks exercise activity, sleep patterns, and heart rate using embedded electronics and is equipped with a camera 202 to capture dietary intake. The wearable device (wristwatch) 200 monitors exercise activity, sleep patterns, and stress using a series of sensors, such as accelerometers, heart rate monitor sensors, etc. Sleep patterns can be determined by considering the heart rate and accelerometer data. The device 200 also includes an advanced microcontroller (processor) running a custom embedded operating system with GUI support. The display 204a and 204b of the device 200 includes a low power LCD screen. The LCD display is selected to support various display options. The device 200 functionality includes: a watch (with timers and medication schedules), exercise activity displayed as number of steps and graphs over period of time, heart rate as average number of beats per minute and graphs over period of time. The device 200 will also equipped with a micro camera module for recording pictures and videos to capture a user's dietary information. The camera 202 has a lens with fixed or varying focal length. The device 200 is also equipped with a low-power LED laser 206 to guide the point of focus of the camera 202. The device 200 also includes infrared (IR) proximity sensor 208 to guide the user to obtain a clear in-focus image of the food they will consume or that they have consumed. The data to and from the device 200 is primarily through the low power Bluetooth (BLE 4.0) communication protocol. The device 200 also supports data transfer to the PC through a micro-USB port. Data exchange and configuration can be done through the micro-USB port. Power management and battery charging is supervised by the microcontroller. The microcontroller will also perform various signal filtering, data logging, and data analysis to determine valid activity and sleep pattern data received from the sensors. The device 200 will also include options to manually enter old-fashioned food diaries and calorie counter. The device 200 can include basic image processing algorithms to identify plates and cups, and determine the amount of food, different types of food, size of the plates and/or cups. The user will be prompted with a series of simple options to determine the calorie intake.

One or more applications running on a computer, mobile phone, electronic tablet or other suitable device can be used to collect, store and visualize data. The application will extract data through Bluetooth communication protocol. The application will be able to collect data and configure the device. A commercial software tool will contain features to store and retrieve the hardware-captured data from the cloud. The software can include advanced image processing algorithms to identify plates and cups, and determine the size of the plate, amount of food, different types of food, size of the cups. The user will be prompted with a series of simple options to determine the calorie intake.

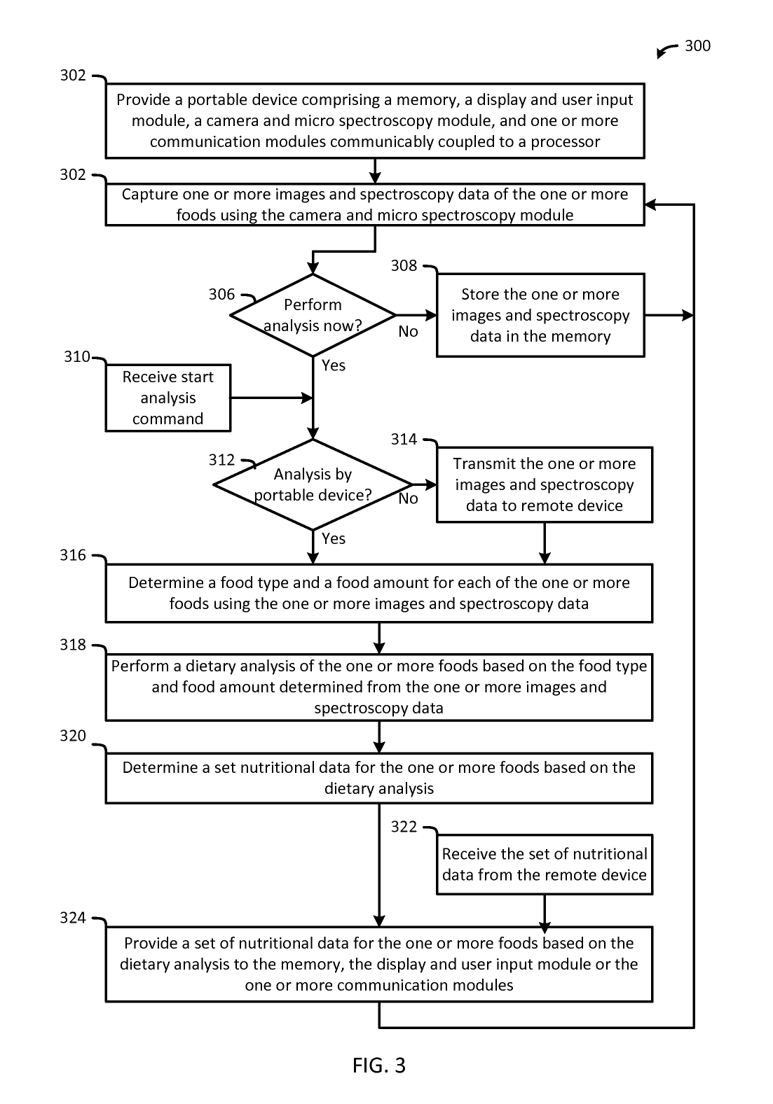

Now referring to FIG. 3, a flow chart of a method 300 for providing a set of nutritional data for one or more foods in accordance with one embodiment of the present invention is shown. A portable device including a memory, a display and user input module, a camera and micro spectroscopy module, and one or more communication modules communicably coupled to a processor is provided in block 302. One or more images and spectroscopy data of the one or more foods are captured using the camera and micro spectroscopy module in block 304. If the analysis is not to be performed now, as determined in decision block 306, the one or more images are stored in memory in block 308 and the process returns to block 304 to capture any additional images and spectroscopy data. If, however, the analysis is to be performed now, as determined in decision block 306 or a start analysis command is received in block 310, the one or more images and spectroscopy data are analyzed by either the portable device or remote device as determined in decision block 312. If the one or more images and spectroscopy data are analyzed by the remote device, as determined in decision block 312, the one or more images and spectroscopy data are transmitted to the remote device in block 314. In either case, a food type and a food amount are determined for each of the one or more foods using the one or more images and the spectroscopy data in block 316, a dietary analysis of the one or more foods is performed based on the food type and food amount determined from the one or more images and spectroscopy data in block 318, and the set of nutritional data for the one or more foods is determined based on the dietary analysis in block 320. If the one or more images and spectroscopy data are analyzed by the remote device, as determined in decision block 312, the set of nutritional data is transmitted to and received by the portable device in block 322. In either case, the set of nutritional data for the one or more foods is provided to the memory, the display and user input module or the one or more communication modules in block 324.

Other steps may also be performed based on the configuration of the portable device, such as: monitoring one or more biological indicators of a user using the one or more sensors; analyzing the one or more biological indicators, and providing a result of the analysis of one or more biological indicators; storing the set of nutritional data; confirming the food type and/or the food amount; requesting an additional information from a user; or other desirable functionality.

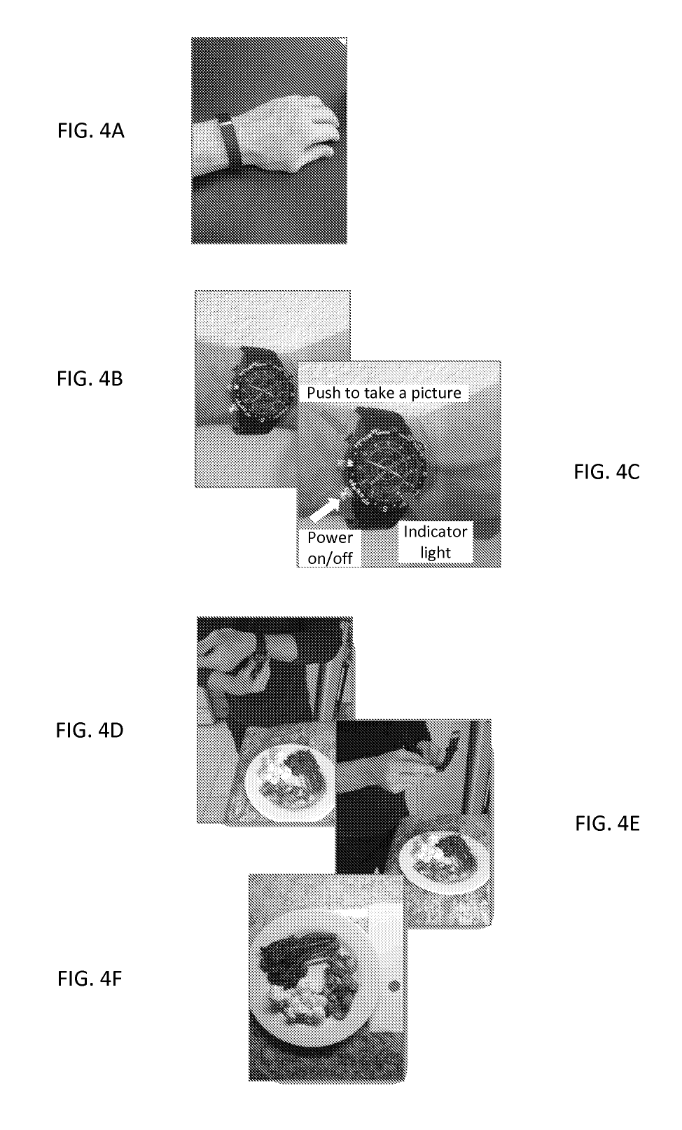

Referring now to FIGS. 4A-4F, photographs illustrating taking one or more images of food in accordance with one embodiment of the present invention are shown.

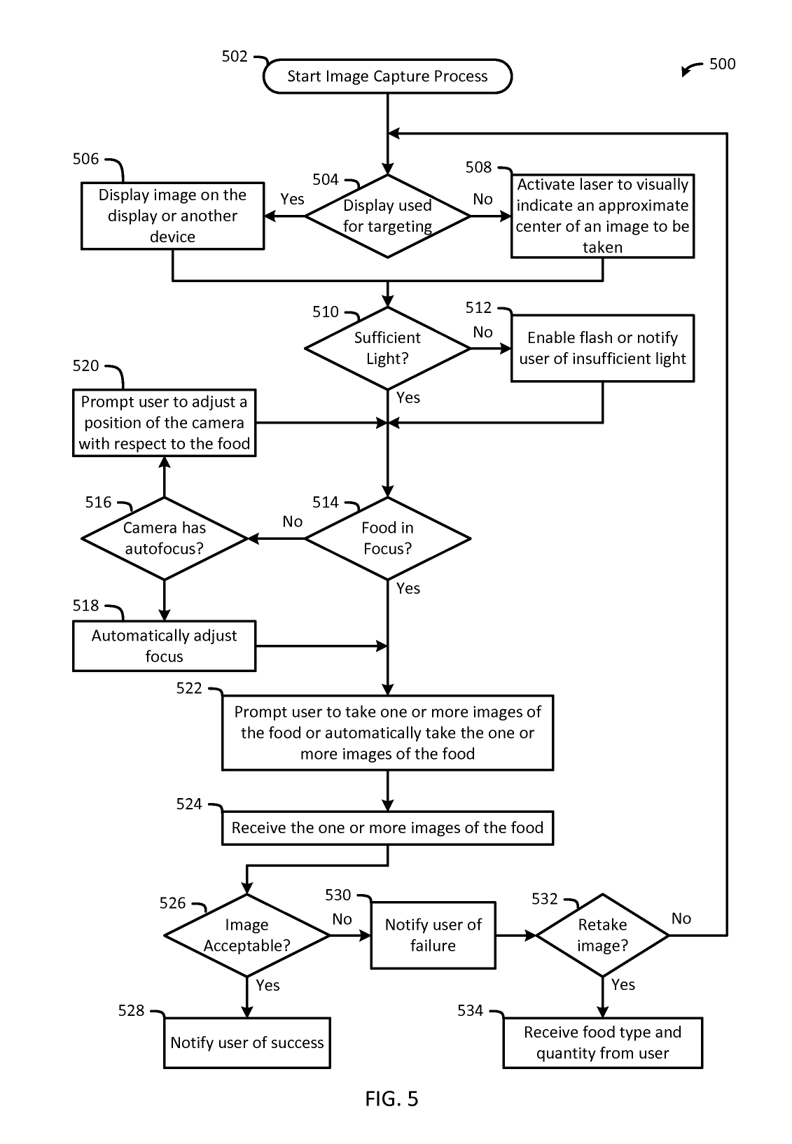

Now referring to FIG. 5, a flow chart of an image capture process 500 in accordance with one embodiment of the present invention is shown. The image capture process 500 starts in block 502. If a display is used to target the camera, as determined in decision block 504, an image of the one or more foods is displayed on the device or on remote device that is in communication with the camera in block 506. If a display is not used to target the camera, as determined in decision block 504, a laser on the device is activated to visually indicate an approximate center of the image to be taken in block 508. Thereafter, if there is not sufficient light to capture a good image of the food, as determined in decision block 510, a flash is enabled or the user is notified that there is insufficient light in block 512. Thereafter, if the food is not in focus, as determined in decision block 514, and the camera has autofocus as determined in decision block 516, the focus is automatically adjusted in block 518. If the food is not in focus, as determined in decision block 514, and the camera does not have autofocus as determined in decision block 516, the user is prompted to adjust a position of the camera with respect to the food to properly focus the image in block 520, and the focus is checked in decision block 514. Once the food is in focus, as determined in decision block 514 or adjusted in block 518, the user is prompted to take one or more images of the food or the camera automatically takes the one or more images of the food in block 522. The processor receives the one or more images of the food from the camera in block 524. If the one or more images are acceptable, as determined in decision block 526, the user is notified in block 528 and the images can be saved or analyzed. If the one or more images are not acceptable, as determined in decision block 526, the user is notified in block 530. If the user desires to retake the images, as determined in decision block 532, the process loops back to decision block 504 where the process is repeated as previously described. If, however, the user does not desire to retake the images, as determined in decision block 532, the user can provide the food type and quantity using a voice or text input in block 534.

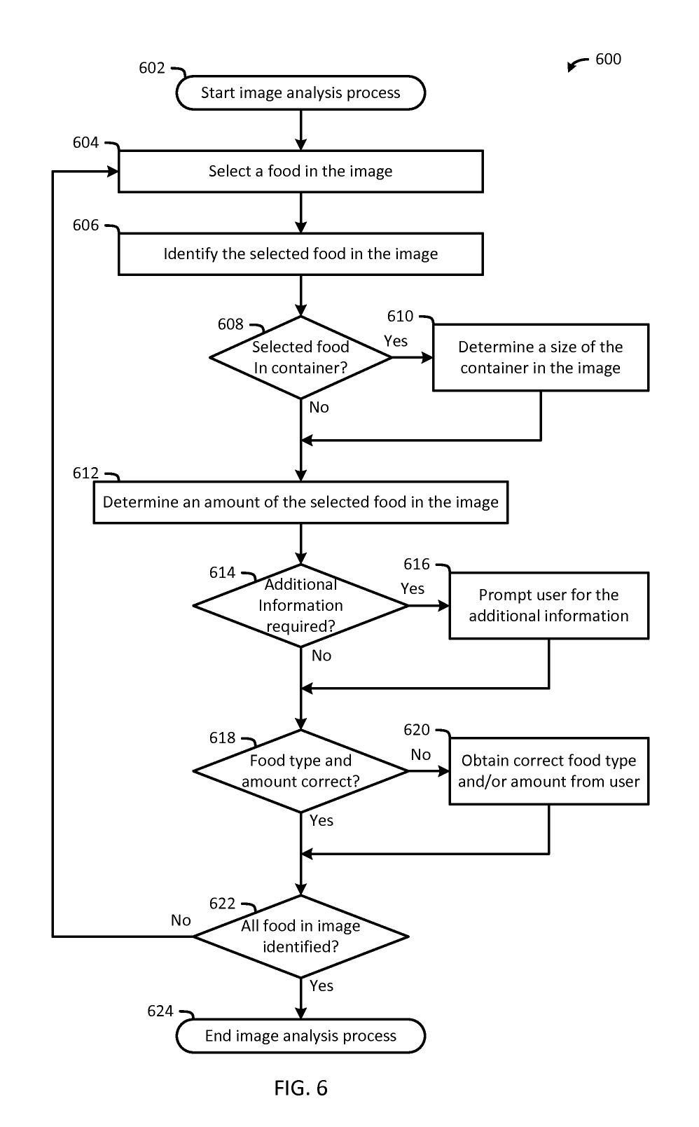

Referring now to FIG. 6, a flow chart of an image analysis process 600 in accordance with one embodiment of the present invention is shown. The image analysis process starts in block 602 and a food is selected in the image in block 604. The selected food in the image is identified in block 606. If the selected food is in a container (e.g., plate, bowl, cup, etc.), as determined in decision block 608, a size of the container in the image is determined in block 610. Thereafter and if the selected food is not in a container, as determined in decision block 608, an amount of the selected food is determined in block 612. If additional information is required, as determined in decision block 614, the user is prompted to provide the additional information in block 616. Thereafter and if additional information is not required, as determined in decision block 614, and the food type and amount are not correct, as determined in decision block 618, the correct food type and/or amount is obtained from the user in block 620. Thereafter and if the food type and amount are correct, as determined in decision block 618, and all the food in the image has been identified, as determined in decision block 622, the image analysis process ends in block 624. If, however, all the food in the image has not been identified, as determined in decision block 622, the process loops back to select another food in the image in block 604 and the process repeats as previously described.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications, patents and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] Subar A F, Kipnis V, Troiano R P, Midthune D, Scoeller D A, Bingham S, Sharbaugh C O, Trabulsi J, Runswick S, Ballard-Barbash R, Sunshine J, Schatzkin A. Using intake biomarkers to evaluate the extent of dietary misreporting in a large sample of adults: The OPEN study. American Journal of Epidemiology. 2003; 158:1-13.
[2] Shai I, Rosner B A, Shahar D R, Vardi H, Azrad A B, Kanfi A, Schwarzfuchs D, Fraser D. Dietary evaluation and attenuation of relative risk: Multiple comparisons between blood and urinary biomarkers, food frequency, and 24-hour recall questionnaires: the DEARR Study. Journal of Nutrition. 2005; 135:573-579.
[3] Tooze J A, Subar A F, Thompson F E, Troiano R, Schatzkin A, Kipnis V. Psychosocial predictors of energy underreporting in a large doubly labeled water study. American Journal of Clinical Nutrition. 2004; 79:795-804.
[4] Bedard D, Shatenstien B, Nadon S. Underreporting of energy intake from a self-administered food-frequency questionnaire completed by adults in Montreal. Public Health Nutrition. 2003; 7:675-681.
[5] Johansson L, Solvoll K, Aa Bjorneboe G E, Drevon C A. Under- and over-reporting of energy intake related to weight status and lifestyle in a nationwide sample. American Journal of Clinical Nutrition. 1998; 68:266-274.
[6] Olafsdottir A S, Thorsdottir I, Gunnarsdottir I, Thorgeirsdottir H, Steingrimsdottir L. Comparison of women's diet assessed by FFQs and 24-hour recalls with and without under-reporters: Associations and biomarkers. Annals of Nutrition & Metabolism. 2006; 50:450-460.
[7] Yannakoulia M, Panagiotakos D B, Pitsavos C, Bathrellou E, Chrysohoou C, Skoumas Y, Stefanadis C. Low energy reporting related to lifestyle, clinical, and pychosocial factors in a randomly selected population sample of Greek adults: The ATTICA study. Journal of the American College of Nutrition. 2007; 26:327-333.
[8] Mayer-Davis E J, Sparks K C, Hirst K, Costacou T, Lovejoy J C, Regensteiner J G, Hoskin M A, Kriska A M, Bray G A, Group TDPPR. Dietary intake in the Diabetes Prevention Program cohort: Baseline and 1-year post-randomization. Annals of Epidemiology. 2004; 14:763-772.
[9] Voss S, Kroke A, Klipstein-Grobusch K, Boeing H. Is macronutrient composition of dietary intake data affected by underreporting? Results from the EPIC-Potsdam Study. European Journal of Clinical Nutrition. 1998; 52:119-126.
[10] Frobisher C, Maxwell S M. The estimation of food portion sizes: a comparison between using descriptions of portion sizes and a photographic food atlas by children and adults. J Hum Nutr Diet. 2003; 16:181-188.
[11] Thompson F E, Subar A, Loria C M, Reedy J L, Baranowski T. Need for technological innovation in dietary assessment. J Am Diet Assoc. 2010 January; 110(1):48-51. doi: 10.1016/j.jada.2009.10.008.

1. A computerized method for providing a set of nutritional data for one or more foods, comprising the steps of:
providing a portable device comprising a memory, a display and user input module, a camera and micro spectroscopy module, and one or more communication modules communicably coupled to a processor;
capturing one or more images and spectroscopy data of the one or more foods using the camera and micro spectroscopy module;
determining a food type and a food amount for each of the one or more foods using the one or more images and spectroscopy data;
performing a dietary analysis of the one or more foods based on the food type and the food amount determined from the one or more images and spectroscopy data;
determining the set of nutritional data for the one or more foods based on the dietary analysis; and
providing the set of nutritional data for the one or more foods to the memory, the display and user input module or the one or more communication modules.

2. The method as recited in claim 1, the portable device further comprising one or more sensors communicably coupled to the processor and the method further comprises the step of monitoring one or more biological indicators of a user using the one or more sensors.

3. The method as recited in claim 2, the one or more biological indicators comprising an exercise activity, a sleep pattern, a stress level, a temperature or a combination thereof.

4. The method as recited in claim 2, the one or more sensors comprising an accelerometer, a heart rate monitor, a thermometer or a combination thereof.

5. The method as recited in claim 2, further comprising the steps of:
analyzing the one or more biological indicators; and
providing a result of the analysis of one or more biological indicators to the memory, the display and user input module or the one or more communication modules.

6. The method as recited in claim 1, further comprising the step of storing the one or more images and spectroscopy data.

7. The method as recited in claim 1, further comprising the step of storing the set of nutritional data.

8. The method as recited in claim 1, further comprising the steps of:
transmitting the one or more images from the portable device to a remote device;
performing the steps of determining the food type and the food amount for each of the one or more foods using the one or more images and spectroscopy data, performing the dietary analysis of the one or more foods based on the food type and the food amount determined from the one or more images and spectroscopy data, and determining the set of nutritional data for the one or more foods based on the dietary analysis using the remote device; and
receiving the set of nutritional data from remote device.

9. The method as recited in claim 8, the remote device comprises a portable computing device, a desktop or laptop computer, a mobile phone, an electronic tablet, or a server computer.

10. The method as recited in claim 1, the portable device further comprising a flash, a laser, an infrared proximity sensor communicably coupled to the processor or the camera.

11. The method as recited in claim 1, the processor having a clock function, a timer function or both.

12. The method as recited in claim 1, the portable device further comprising a global positioning module communicably coupled to the processor.

13. The method as recited in claim 1, further comprising a battery.

14. The method as recited in claim 11, the battery further comprising a recharging port connected to the battery, or a battery recharger connected to the battery that recharges the battery using electromagnetic fields, motion or solar energy.

15. The method as recited in claim 1, the display and user input module comprises one or more displays, one or more buttons, one or more touch screen, or a combination thereof.

16. The method as recited in claim 1, the one or more communication modules comprising a wireless communication module, an infrared module, a cable connector, or a combination thereof.

17. The method as recited in claim 1, further comprising the step of confirming the food type and the food amount.

18. The method as recited in claim 1, further comprising the step of requesting an additional information from a user.

19. An apparatus for providing a set of nutritional data for one or more foods comprising:
a portable device housing;
a processor disposed within the portable device housing;
a memory disposed within the portable device housing and communicably coupled to the processor;
a display and user input module disposed on the portable device housing and communicably coupled to the processor;
a camera disposed on the portable device housing and communicably coupled to the processor;
a near infrared spectroscopy module disposed on the portable device housing and communicably coupled to the processor;
one or more communication modules disposed on or within the portable device housing and communicably coupled to the processor; and
the processor configured to capture one or more images of the one or more foods using the camera and a near infrared spectroscopy data of the one or more foods using the near infrared spectroscopy module, determine a food type and a food amount for each of the one or more foods using the one or more images and near infrared spectroscopy data, perform a dietary analysis of the one or more foods based on the food type and food amount determined from the one or more images and the near infrared spectroscopy data, determine the set of nutritional data for the one or more foods based on the dietary analysis, and provide the set of nutritional data for the one or more foods to the memory, the display and user input module or the one or more communication modules.

20. The apparatus as recited in claim 19, further comprising one or more sensors disposed on or within the portable device housing and communicably coupled to the processor, and the processor is further configured to monitor one or more biological indicators of a user using the one or more sensors.

21. The apparatus as recited in claim 20, the one or more biological indicators comprising an exercise activity, a sleep pattern, a stress level, a temperature or a combination thereof.

22. The apparatus as recited in claim 20, the one or more sensors comprising an accelerometer, a heart rate monitor, a thermometer or a combination thereof.

23. The apparatus as recited in claim 20, the processor further configured to analyze the one or more biological indicators and provide a result of the analysis of one or more biological indicators to the memory, the display and user input module or the one or more communication modules.

24. The apparatus as recited in claim 19, the processor further configured to store the one or more images and the near infrared spectroscopy data.

25. The apparatus as recited in claim 19, the processor further configured to store the set of nutritional data.

26. The apparatus as recited in claim 19, the processor further configured to transmit the one or more images and the near infrared spectroscopy data to a remote device, and receive the set of nutritional data from the remote device, wherein the remote device is configured to determine the food type and the food amount for each of the one or more foods using the one or more images and the near infrared spectroscopy data, perform a dietary analysis of the one or more foods based on the food type and food amount determined from the one or more images and the near infrared spectroscopy data, and determine the set of nutritional data for the one or more foods based on the dietary analysis.

27. The apparatus as recited in claim 26, the remote device comprises a portable computing device, a desktop or laptop computer, a mobile phone, an electronic tablet, or a server computer.

28. The apparatus as recited in claim 19, further comprising a flash, a laser, an infrared proximity sensor disposed on the portable device housing and communicably coupled to the processor or the camera.

29. The apparatus as recited in claim 19, the processor having a clock function, a timer function or both.

30. The apparatus as recited in claim 19, further comprising a global positioning module disposed within the portable device housing and communicably coupled to the processor.

31. The apparatus as recited in claim 19, further comprising a battery disposed within the portable device housing.

32. The apparatus as recited in claim 31, the battery further comprising a recharging port connected to the battery, or a battery recharger connected to the battery that recharges the battery using electromagnetic fields, motion or solar energy.

33. The apparatus as recited in claim 19, the display and user input module comprises one or more displays, one or more buttons, one or more touch screen, or a combination thereof.

34. The apparatus as recited in claim 19, the one or more communication modules comprising a wireless communication interface, an infrared module, a cable connector, or a combination thereof.

35. The apparatus as recited in claim 19, the processor further configured to confirm the food type and the food amount.

36. The apparatus as recited in claim 19, the processor further configured to request an additional information from a user.
